(19) United States
(12) Patent Application Publication
(10) Pub. No.: US 2023/0281754 A1
(43) Pub. Date: Sep. 7, 2023

(54) IMAGING METHODS USING AN IMAGE SENSOR WITH MULTIPLE RADIATION DETECTORS

(71) Applicant: SHENZHEN XPECTVISION TECHNOLOGY CO., LTD., Shenzhen (CN)

(72) Inventors: Yurun LIU, Shenzhen (CN); Peiyan CAO, Shenzhen (CN)

(21) Appl. No.: 18/196,010

(22) Filed: May 11, 2023

Related U.S. Application Data
(63) Continuation of application No. PCT/CN2020/131473, filed on Nov. 25, 2020.

(51) Int. Cl. G06T 3/40

(52) U.S. Cl. CPC G06T 3/4038; G06T 3/4053

(57) ABSTRACT

Disclosed herein is a method, comprising (A) shining a scene with radiation pulses (i), i=1, . . . , M, one pulse at a time, wherein M is an integer greater than 1; (B) for i=1, . . . , M, during the radiation pulse (i) and utilizing radiation of the radiation pulse (i), capturing, one by one, partial images (i,j), j=1, . . . , Ni of the scene with a same image sensor, wherein Ni, i=1, . . . , M are all integers greater than 1; (C) for i=1, . . . , M, generating an enhanced partial image (i) from the partial images (i,j), j=1, . . . , Ni by applying one or more super resolution algorithms to the partial images (i,j), j=1, . . . , Ni; and (D) stitching the enhanced partial images (i), i=1, . . . , M resulting in a stitched image of the scene.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray, or γ-ray. The radiation may be of other types such as α-rays and βrays. An imaging system may include an image sensor having multiple radiation detectors.

### SUMMARY

Disclosed herein is a method, comprising shining a scene with radiation pulses (i), i=1, . . . , M, one pulse at a time, wherein M is an integer greater than 1; for i=1, . . . , M, during the radiation pulse (i) and utilizing radiation of the radiation pulse (i), capturing, one by one, partial images (i,j), j=1, . . . , Ni of the scene with a same image sensor, wherein Ni, i=1, . . . , M are all integers greater than 1; for i=1, . . . , M, generating an enhanced partial image (i) from the partial images (i,j), j=1, . . . , Ni by applying one or more super resolution algorithms to the partial images (i,j), j=1, . . . , Ni; and stitching the enhanced partial images (i), i=1, . . . , M resulting in a stitched image of the scene.

In an aspect, all Ni, i=1, . . . , M are the same.

In an aspect, all Ni, i=1, . . . , M are greater than 100.

In an aspect, for i=1, . . . , M, during the radiation pulse (i), the image sensor moves continuously with respect to the scene.

In an aspect, the image sensor moves continuously with respect to the scene during a time period in which the image sensor captures all the partial images (i,j), i=1, . . . , M, and j=1, . . . , Ni.

In an aspect, said moving of the image sensor with respect to the scene during the time period is at a constant speed.

In an aspect, the method further comprises arranging a mask such that for i=1, . . . , M, during the radiation pulse (i), (A) radiation of the radiation pulse (i) which is aimed at the scene but not aimed at active areas of the image sensor is prevented by the mask from reaching the scene, and (B) radiation of the radiation pulse (i) which is aimed at the scene and also aimed at the active areas of the image sensor is allowed by the mask to pass through the mask so as to reach the scene.

In an aspect, during each of the radiation pulses (i), i=1, . . . , M, the image sensor moves a distance of less than a width of a sensing element of the image sensor measured in a direction of said moving of the image sensor.

In an aspect, during each of the radiation pulses (i), i=1, . . . , M, the image sensor moves a distance of less than one half of said width.

In an aspect, the image sensor comprises multiple radiation detectors.

Disclosed herein is an imaging system, comprising a radiation source configured to shine a scene with radiation pulses (i), i=1, . . . , M, one pulse at a time, wherein M is an integer greater than 1; and an image sensor configured to, for i=1, . . . , M, during the radiation pulse (i) and utilizing radiation of the radiation pulse (i), capture one by one, partial images (i,j), j=1, . . . , Ni of the scene, wherein Ni, i=1, . . . , M are all integers greater than 1, wherein the image sensor is configured to, for i=1, . . . , M, generate an enhanced partial image (i) from the partial images (i,j), j=1, . . . , Ni by applying one or more super resolution algorithms to the partial images (i,j), j=1, . . . , Ni, and wherein the image sensor is configured to stitch the enhanced partial images (i), i=1, . . . , M resulting in a stitched image of the scene.

In an aspect, all Ni, i=1, . . . , M are the same.

In an aspect, all Ni, i=1, . . . , M are greater than 100.

In an aspect, for i=1, . . . , M, during the radiation pulse (i), the image sensor is configured to move continuously with respect to the scene.

In an aspect, the image sensor is configured to move continuously with respect to the scene during a time period in which the image sensor captures all the partial images (i,j), i=1, . . . , M, and j=1, . . . , Ni.

In an aspect, said moving of the image sensor with respect to the scene during the time period is at a constant speed.

In an aspect, the imaging system further comprises a mask arranged such that for i=1, . . . , M, during the radiation pulse (i), (A) radiation of the radiation pulse (i) which is aimed at the scene but not aimed at active areas of the image sensor is prevented by the mask from reaching the scene, and (B) radiation of the radiation pulse (i) which is aimed at the scene and also aimed at the active areas of the image sensor is allowed by the mask to pass through the mask so as to reach the scene.

In an aspect, during each of the radiation pulses (i), i=1, . . . , M, the image sensor is configured to move a distance of less than a width of a sensing element of the image sensor measured in a direction of said moving of the image sensor.

In an aspect, during each of the radiation pulses (i), i=1, . . . , M, the image sensor is configured to move a distance of less than one half of said width.

In an aspect, the image sensor comprises multiple radiation detectors.

### BRIEF DESCRIPTION OF FIGURES

FIG. 1 schematically shows a radiation detector, according to an embodiment.

FIG. 2A schematically shows a simplified cross-sectional view of the radiation detector, according to an embodiment.

FIG. 2B schematically shows a detailed cross-sectional view of the radiation detector, according to an embodiment.

FIG. 2C schematically shows a detailed cross-sectional view of the radiation detector, according to an alternative embodiment.

FIG. 3 schematically shows a top view of a package including the radiation detector and a printed circuit board (PCB), according to an embodiment.

FIG. 4 schematically shows a cross-sectional view of an image sensor including a plurality of the packages of FIG. 3 mounted to a system PCB (printed circuit board), according to an embodiment.

FIG. 5A-FIG. 5G show an image sensor going though an imaging session, according to an embodiment.

FIG. 6 shows a flowchart summarizing and generalizing the imaging session described in FIG. 5A-FIG. 5G.

FIG. 7 shows a mask used with the image sensor of FIG. 5A-FIG. 5G, according to an embodiment.

### DETAILED DESCRIPTION

### RADIATION DETECTOR

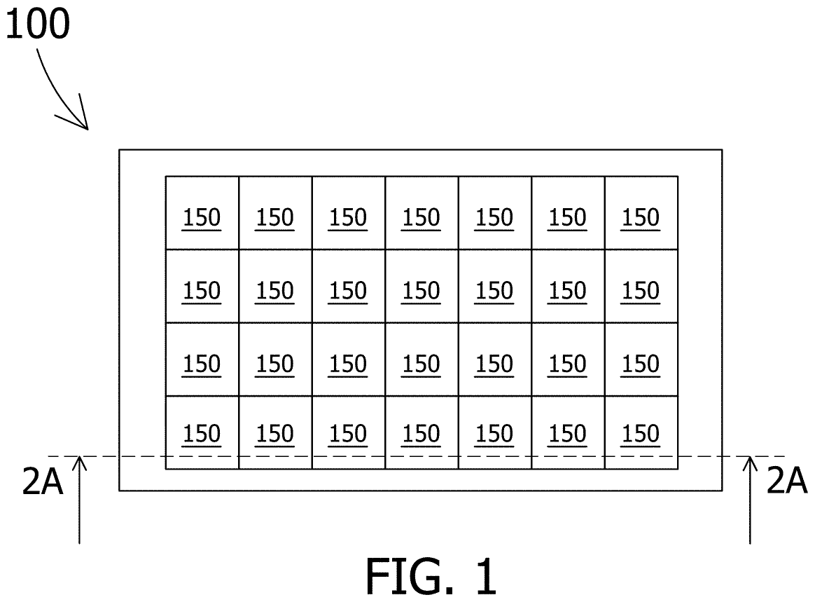

FIG. 1 schematically shows a radiation detector 100, as an example. The radiation detector 100 may include an array of pixels 150 (also referred to as sensing elements 150). The array may be a rectangular array (as shown in FIG. 1), a honeycomb array, a hexagonal array, or any other suitable array. The array of pixels 150 in the example of FIG. 1 has 4 rows and 7 columns; however, in general, the array of pixels 150 may have any number of rows and any number of columns.

Each pixel 150 may be configured to detect radiation from a radiation source (not shown) incident thereon and may be configured to measure a characteristic (e.g., the energy of the particles, the wavelength, and the frequency) of the radiation. A radiation may include particles such as photons and subatomic particles. Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

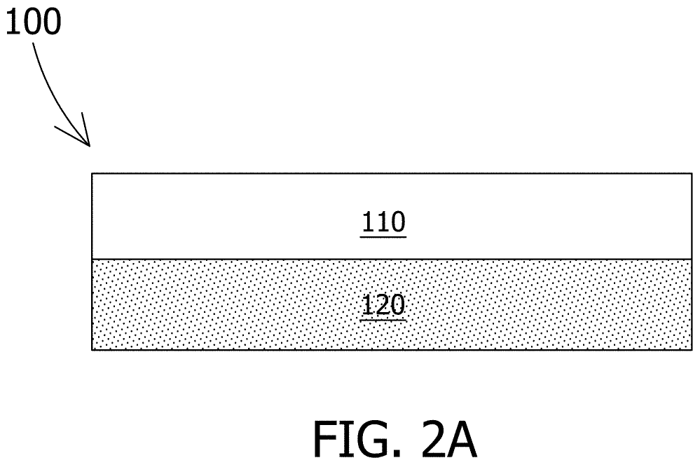

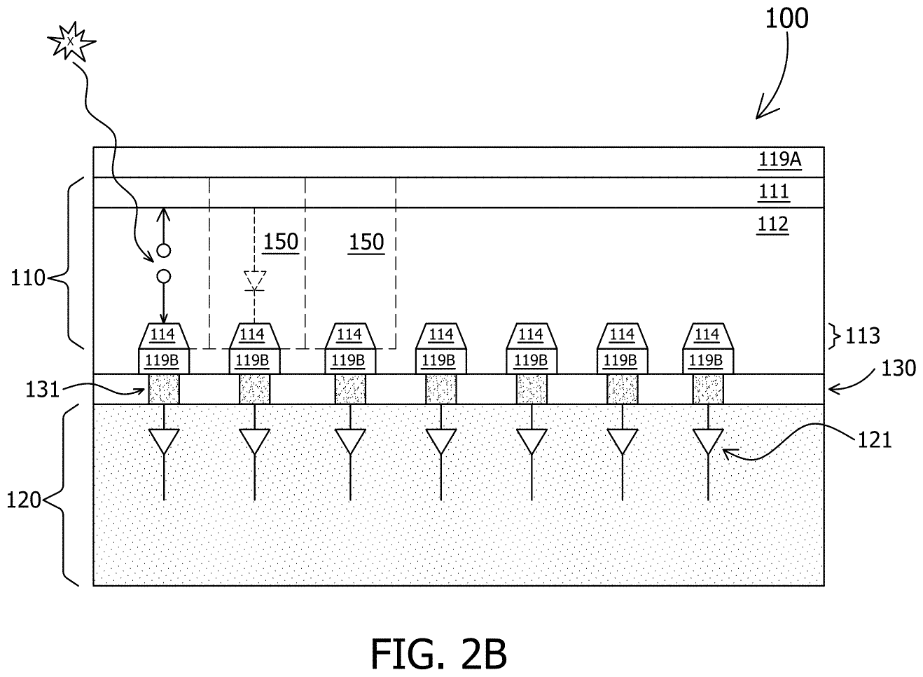

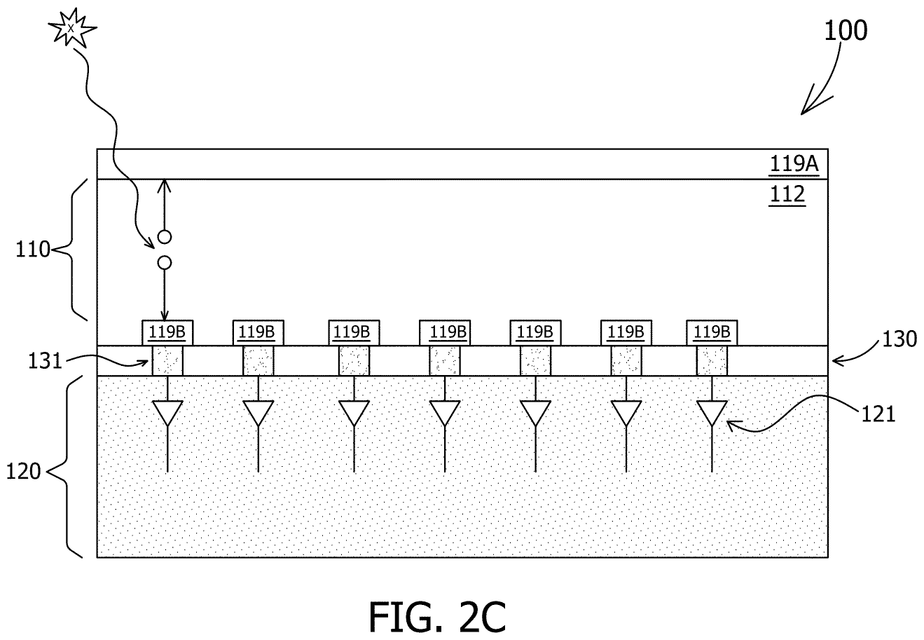

FIG. 2A schematically shows a simplified cross-sectional view of the radiation detector 100 of FIG. 1 along a line 2A-2A, according to an embodiment. More specifically, the radiation detector 100 may include a radiation absorption layer 110 and an electronics layer 120 (e.g., an ASIC or application-specific integrated circuit) for processing or analyzing electrical signals which incident radiation generates in the radiation absorption layer 110. The radiation detector 100 may or may not include a scintillator (not shown). The radiation absorption layer 110 may include a semiconductor material such as silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. More specifically, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional intrinsic region 112. The discrete regions 114 may be separated from one another by the first doped region 111 or the intrinsic region 112. The first doped region 111 and the second doped region 113 may have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, 7 diodes corresponding to 7 pixels 150 of one row in the array of FIG. 1, of which only 2 pixels 150 are labeled in FIG. 2B for simplicity). The plurality of diodes may have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs (analog to digital converters). The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The electric field may be an external electric field. The electrical contact 1198 may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows a detailed cross-sectional view of the radiation detector 100 of FIG. 1 along the line 2A-2A, according to an alternative embodiment. More specifically, the radiation absorption layer 110 may include a resistor of a semiconductor material such as silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C is similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B may include discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be an area around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

### Radiation Detector Package

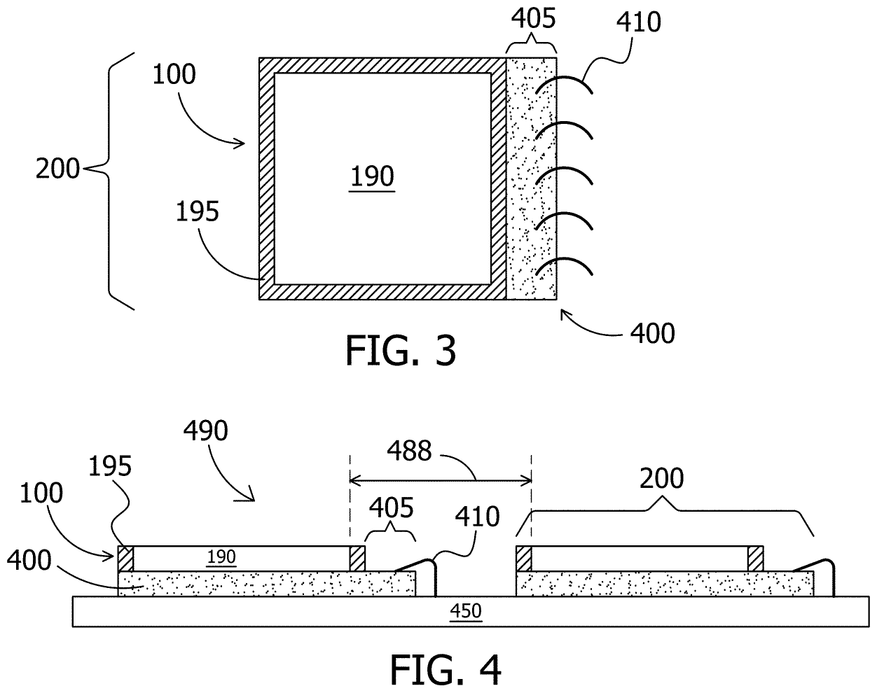

FIG. 3 schematically shows a top view of a package 200 including the radiation detector 100 and a printed circuit board (PCB) 400. The term “PCB” as used herein is not limited to a particular material. For example, a PCB may include a semiconductor. The radiation detector 100 may be mounted to the PCB 400. The wiring between the radiation detector 100 and the PCB 400 is not shown for the sake of clarity. The PCB 400 may have one or more radiation detectors 100. The PCB 400 may have an area 405 not covered by the radiation detector 100 (e.g., for accommodating bonding wires 410). The radiation detector 100 may have an active area 190 which is where the pixels 150 (FIG. 1) are located. The radiation detector 100 may have a perimeter zone 195 near the edges of the radiation detector 100. The perimeter zone 195 has no pixels 150, and the radiation detector 100 does not detect particles of radiation incident on the perimeter zone 195.

### Image Sensor

FIG. 4 schematically shows a cross-sectional view of an image sensor 490, according to an embodiment. The image sensor 490 may include a plurality of the packages 200 of FIG. 3 mounted to a system PCB 450. FIG. 4 shows only 2 packages 200 as an example. The electrical connection between the PCBs 400 and the system PCB 450 may be made by bonding wires 410. In order to accommodate the bonding wires 410 on the PCB 400, the PCB 400 may have the area 405 not covered by the radiation detector 100. In order to accommodate the bonding wires 410 on the system PCB 450, the packages 200 may have gaps in between. The gaps may be approximately 1 mm or more. Particles of radiation incident on the perimeter zones 195, on the area 405, or on the gaps cannot be detected by the packages 200 on the system PCB 450. A dead zone of a radiation detector (e.g., the radiation detector 100) is the area of the radiation-receiving surface of the radiation detector, on which incident particles of radiation cannot be detected by the radiation detector. A dead zone of a package (e.g., package 200) is the area of the radiation-receiving surface of the package, on which incident particles of radiation cannot be detected by the radiation detector or detectors in the package. In this example shown in FIG. 3 and FIG. 4, the dead zone of the package 200 includes the perimeter zones 195 and the area 405. A dead zone (e.g., 488) of an image sensor (e.g., image sensor 490) with a group of packages (e.g., packages 200 mounted on the same PCB, packages 200 arranged in the same layer) includes the combination of the dead zones of the packages in the group and the gaps between the packages.

The image sensor 490 including the radiation detectors 100 may have the dead zone 488 incapable of detecting incident radiation. However, the image sensor 490 may capture partial images of all points of an object or scene (not shown), and then these captured partial images may be stitched to form an image of the entire object or scene.

### Imaging Session

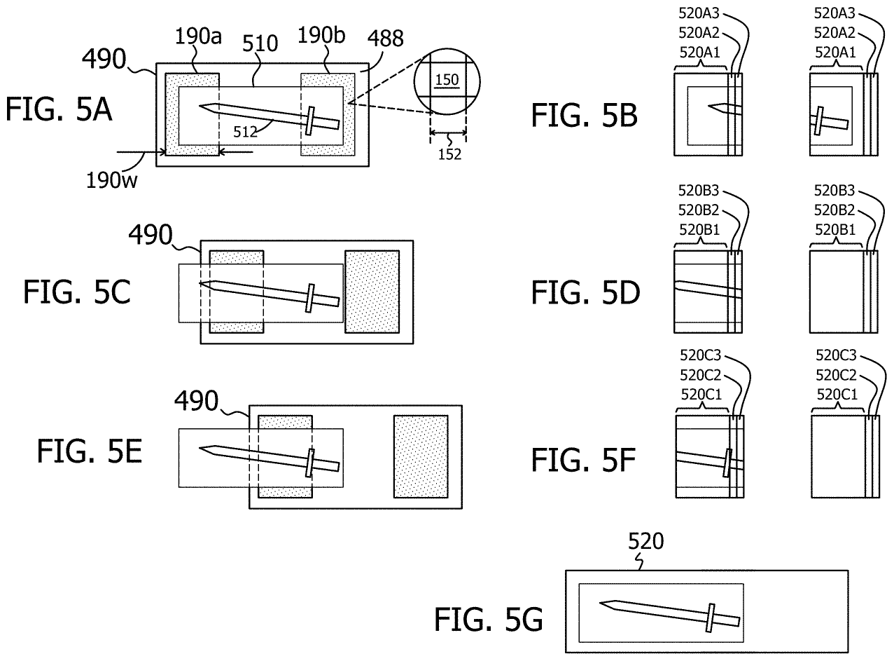

FIG. 5A-FIG. 5G show the image sensor 490 of FIG. 4 going though an imaging session, according to an embodiment. For simplicity, only active areas 190a and 190b and the dead zone 488 of the image sensor 490 are shown (i.e., other details of the image sensor 490 are omitted).

In an embodiment, during the imaging session, the image sensor 490 may move from left to right while an object (or scene) 510 remains stationary as the image sensor 490 scans the object 510. For example, the object 510 may be a cardboard box containing a sword 512.

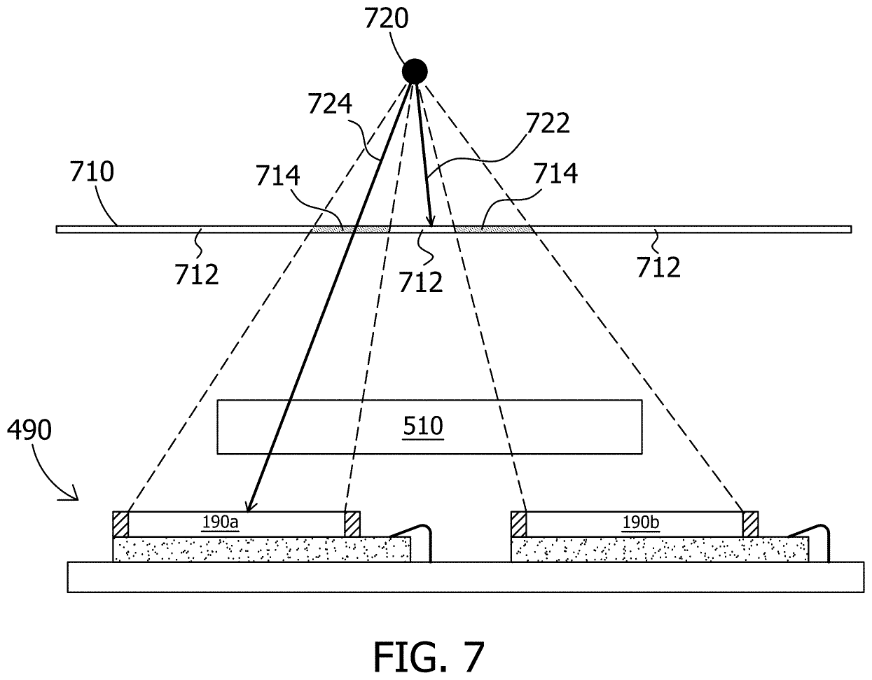

In an embodiment, during the imaging session, a radiation source 720 (FIG. 7, but not shown in FIG. 5A-FIG. 5G for simplicity) may send radiation through the object 510 to the image sensor 490. In other words, the object 510 is positioned between the radiation source 720 and the image sensor 490.

In an embodiment, the imaging session may start with the image sensor 490 moving to the right to a first imaging position as shown in FIG. 5A. At the first imaging position, using the radiation from the radiation source 720, the image sensor 490 may capture a partial image 520A1 (FIG. 5B) of the object 510.

Next, in an embodiment, the image sensor 490 may move further to the right by a short distance (e.g., less than the size of a pixel 150 of the image sensor 490) to a second imaging position (not shown). At the second imaging position, using the radiation from the radiation source 720, the image sensor 490 may capture a partial image 520A2 (FIG. 5B) of the object 510. In FIG. 5B, for comparison, the partial images 520A1 and 520A2 are aligned such that the images of the object 510 in the partial images 520A1 and 520A2 coincide. For simplicity, only the portion of the partial image 520A2 that does not overlap the partial image 520A1 is shown.

Next, in an embodiment, the image sensor 490 may move further to the right by a short distance (e.g., less than the size of a pixel 150 of the image sensor 490) to a third imaging position (not shown). At the third imaging position, using the radiation from the radiation source 720, the image sensor 490 may capture a partial image 520A3 (FIG. 5B) of the object 510. In FIG. 5B, for comparison, the partial images 520A2 and 520A3 are aligned such that the images of the object 510 in the partial images 520A2 and 520A3 coincide. For simplicity, only the portion of the partial image 520A3 that does not overlap the partial image 520A2 is shown.

Next, in an embodiment, the image sensor 490 may move further to the right by a long distance (e.g., about the width 190w (FIG. 5A) of the active area 190a) to a fourth imaging position as shown in FIG. 5C. At the fourth imaging position, using the radiation from the radiation source 720, the image sensor 490 may capture a partial image 520B1 (FIG. 5D) of the object 510.

Next, in an embodiment, the image sensor 490 may move further to the right by a short distance (e.g., less than the size of a pixel 150 of the image sensor 490) to a fifth imaging position (not shown). At the fifth imaging position, using the radiation from the radiation source 720, the image sensor 490 may capture a partial image 520B2 (FIG. 5D) of the object 510. In FIG. 5D, for comparison, the partial images 520B1 and 520B2 are aligned such that the images of the object 510 in the partial images 520B1 and 520B2 coincide. For simplicity, only the portion of the partial image 520B2 that does not overlap the partial image 520B1 is shown.

Next, in an embodiment, the image sensor 490 may move further to the right by a short distance (e.g., less than the size of a pixel 150 of the image sensor 490) to a sixth imaging position (not shown). At the sixth imaging position, using the radiation from the radiation source 720, the image sensor 490 may capture a partial image 520B3 (FIG. 5D) of the object 510. In FIG. 5D, for comparison, the partial images 520B2 and 520B3 are aligned such that the images of the object 510 in the partial images 520B2 and 520B3 coincide. For simplicity, only the portion of the partial image 520B3 that does not overlap the partial image 520B2 is shown.

Next, in an embodiment, the image sensor 490 may move further to the right by a long distance (e.g., about the width 190w (FIG. 5A) of the active area 190a) to a seventh imaging position as shown in FIG. 5E. At the seventh imaging position, using the radiation from the radiation source 720, the image sensor 490 may capture a partial image 520C1 (FIG. 5F) of the object 510.

Next, in an embodiment, the image sensor 490 may move further to the right by a short distance (e.g., less than the size of a pixel 150 of the image sensor 490) to an eighth imaging position (not shown). At the eighth imaging position, using the radiation from the radiation source 720, the image sensor 490 may capture a partial image 520C2 (FIG. 5F) of the object 510. In FIG. 5F, for comparison, the partial images 520C1 and 520C2 are aligned such that the images of the object 510 in the partial images 520C1 and 520C2 coincide. For simplicity, only the portion of the partial image 520C2 that does not overlap the partial image 520C1 is shown.

Next, in an embodiment, the image sensor 490 may move further to the right by a short distance (e.g., less than the size of a pixel 150 of the image sensor 490) to a ninth imaging position (not shown). At the ninth imaging position, using the radiation from the radiation source 720, the image sensor 490 may capture a partial image 520C3 (FIG. 5F) of the object 510. In FIG. 5F, for comparison, the partial images 520C2 and 520C3 are aligned such that the images of the object 510 in the partial images 520C2 and 520C3 coincide. For simplicity, only the portion of the partial image 520C3 that does not overlap the partial image 520C2 is shown.

In an embodiment, throughout the imaging session during which the 9 partial images 520A1, 520A2, 520A3, 520B1, 520B2, 520B3, 520C1, 520C2, and 520C3 are captured, the radiation source may shine the image sensor 490 and the object 510 with radiation all the time. In an alternative embodiment, during the imaging session, the radiation source 720 may shine the image sensor 490 and the object 510 with radiation in pulses. Specifically, during each pulse, the radiation source 720 shines the image sensor 490 and the object 510 with radiation. However, between the pulses, the radiation source 720 does not shine the image sensor 490 and the object 510 with radiation. In an embodiment, this may be implemented by keeping the radiation source 720 off between the pulses and on during the pulses.

In an embodiment, a first radiation pulse may start before the image sensor 490 captures the partial image 520A1 and end after the image sensor 490 captures the partial image 520A3. In other words, the image sensor 490 captures the partial images 520A1, 520A2, and 520A3 during the first radiation pulse.

In an embodiment, a second radiation pulse may start before the image sensor 490 captures the partial image 520B1 and end after the image sensor 490 captures the partial image 520B3. In other words, the image sensor 490 captures the partial images 520B1, 520B2, and 520B3 during the second radiation pulse.

In an embodiment, a third radiation pulse may start before the image sensor 490 captures the partial image 520C1 and end after the image sensor 490 captures the partial image 520C3. In other words, the image sensor 490 captures the partial images 520C1, 520C2, and 520C3 during the third radiation pulse.

In an embodiment, a first enhanced partial image (not shown) of the object 510 may be generated from the partial images 520A1, 520A2, and 520A3. In an embodiment, one or more super resolution algorithms may be applied to the partial images 520A1, 520A2, and 520A3 so as to generate the first enhanced partial image. In an embodiment, the one or more super resolution algorithms may be applied to the partial images 520A1, 520A2, and 520A3 by the image sensor 490.

In an embodiment, similarly, a second enhanced partial image (not shown) of the object 510 may be generated from the partial images 520B1, 520B2, and 520B3. In an embodiment, one or more super resolution algorithms may be applied to the partial images 520B1, 520B2, and 520B3 so as to generate the second enhanced partial image. In an embodiment, the one or more super resolution algorithms may be applied to the partial images 520B1, 520B2, and 520B3 by the image sensor 490.

In an embodiment, similarly, a third enhanced partial image (not shown) of the object 510 may be generated from the partial images 520C1, 520C2, and 520C3. In an embodiment, one or more super resolution algorithms may be applied to the partial images 520C1, 520C2, and 520C3 so as to generate the third enhanced partial image. In an embodiment, the one or more super resolution algorithms may be applied to the partial images 520C1, 520C2, and 520C3 by the image sensor 490.

In an embodiment, the first enhanced partial image, the second enhanced partial image, and the third enhanced partial image of the object 510 may be stitched to form a stitched image 520 (FIG. 5G) of the object 510. In an embodiment, the stitching of the first, second, and third enhanced partial images may be performed by the image sensor 490.

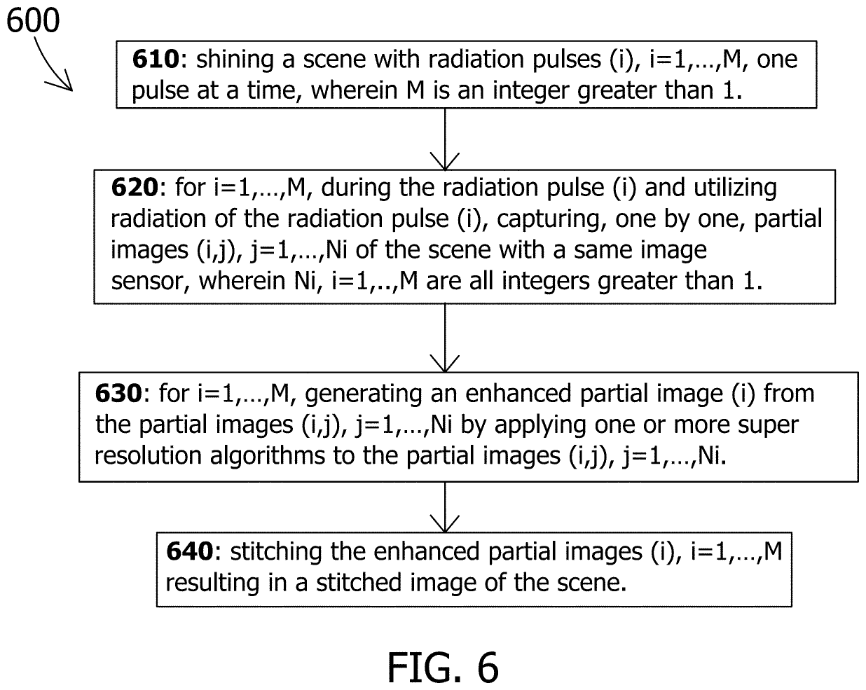

FIG. 6 shows a flowchart 600 summarizing and generalizing the imaging session described above, according to an embodiment. In step 610, a scene may be shined with radiation pulses (i), i=1, . . . , M, one pulse at a time, wherein M is an integer greater than 1. For example, the object or scene 510 of FIG. 5A-FIG. 5E is shined with the first, second, and then third radiation pulses (i.e., M=3).

In step 620, for i=1, . . . , M, during the radiation pulse (i) and utilizing radiation of the radiation pulse (i), partial images (i,j), j=1, . . . , Ni of the scene may be captured one by one with a same image sensor, wherein Ni, i=1, . . . , M are all integers greater than 1. For example, for i=1, during the first radiation pulse and utilizing radiation of the first radiation pulse, the partial images 520A1, 520A2, and 520A3 are captured one by one with the image sensor 490. For i=2, during the second radiation pulse and utilizing radiation of the second radiation pulse, the partial images 520B1, 520B2, and 520B3 are captured one by one with the image sensor 490. For i=3, during the third radiation pulse and utilizing radiation of the third radiation pulse, the partial images 520C1, 520C2, and 520C3 are captured one by one with the image sensor 490.

In step 630, for i=1, . . . , M, an enhanced partial image (i) may be generated from the partial images (i,j), j=1, . . . , Ni by applying one or more super resolution algorithms. For example, for i=1, the first enhanced partial image is generated from the partial images 520A1, 520A2, and 520A3 by applying one or more super resolution algorithms to the partial images 520A1, 520A2, and 520A3. For i=2, the second enhanced partial image is generated from the partial images 520B1, 520B2, and 520B3 by applying one or more super resolution algorithms to the partial images 520B1, 520B2, and 520B3. For i=3, the third enhanced partial image is generated from the partial images 520C1, 520C2, and 520C3 by applying one or more super resolution algorithms to the partial images 520C1, 520C2, and 520C3.

In step 640, the enhanced partial images (i), i=1, . . . , M may be stitched resulting in a stitched image of the scene. For example, the first, second, and third enhanced partial images are stitched resulting in the stitched image 520 (FIG. 5G) of the scene or object 510.

In an embodiment, with respect to step 620 of the flowchart 600 of FIG. 6, all Ni, i=1, . . . , M may be the same. In the embodiments described above, N1=N2=N3=3. In other words, the image sensor 490 captures the same number of partial images of the object 510 during each radiation pulse. In an embodiment, all Ni, i=1, . . . , M may be greater than 100. In general, all Ni, i=1, . . . , M are not necessarily the same. For example, instead of N1=N2=N3=3 as in the embodiments described above, it may be that N1=2, N2=3, and N3=5.

In an embodiment, with respect to the flowchart 600 of FIG. 6, for i=1, . . . , M, during the radiation pulse (i), the image sensor 490 may move continuously (i.e., non-stop) with respect to the scene or object 510.

In an embodiment, with respect to FIG. 5A-FIG. 5E, the image sensor 490 may move continuously (i.e., non-stop) with respect to the object 510 during the entire imaging session. In other words, the image sensor 490 moves continuously with respect to the object 510 during a time period in which the image sensor 490 captures the partial images 520A1, 520A2, 520A3, 520B1, 520B2, 520B3, 520C1, 520C2, and 520C3. With respect to the flowchart 600 of FIG. 6, this means that the image sensor 490 moves continuously (i.e., non-stop) with respect to the object 510 during a time period in which the image sensor 490 captures all the partial images (i,j), i=1, . . . , M, and j=1, . . . , Ni. In an embodiment, the movement of the image sensor 490 with respect to the object 510 during the entire imaging session (i.e., during the time period in which the image sensor 490 captures all the partial images (i,j), i=1, . . . , M, and j=1, . . . , Ni) may be at a constant speed.

In an embodiment, with reference to FIG. 5A-FIG. 5E, and FIG. 7, a mask 710 may be positioned between the object 510 and the radiation source 720. During the imaging session, the mask 710 may be moved with respect to the object 510 and along with the image sensor 490 such that (A) radiation of each radiation pulse of the radiation source 720 which is aimed at the object 510 but not aimed at the active areas 190a and 190b of the image sensor 490 is prevented by the mask 710 from reaching the object 510, and (B) radiation of each radiation pulse of the radiation source 720 which is aimed at the object 510 and also aimed at the active areas 190a and 190b of the image sensor 490 is allowed by the mask 710 to pass through the mask 710 so as to reach the object 510.

For example, a radiation ray 722 which is aimed at the object 510 but not aimed at the active areas 190a and 190b of the image sensor 490 is prevented by a radiation blocking region 712 of the mask 710 from reaching the object 510. For another example, a radiation ray 724 which is aimed at the object 510 and also aimed at the active areas 190a and 190b of the image sensor 490 is allowed by a radiation passing region 714 of the mask 710 to pass through the mask 710 so as to reach the object 510.

In an embodiment, the distance between the first and third imaging positions may be less than a width 152 (FIG. 5A) of a pixel 150 of the image sensor 490 measured in the direction of the movement of the image sensor 490 with respect to the object 510. Similarly, the distance between the fourth and sixth imaging positions may be less than the width 152 (FIG. 5A). Similarly, the distance between the seventh and ninth imaging positions may be less than the width 152 (FIG. 5A). In other words, with respect to the flowchart 600 of FIG. 6, during each of the radiation pulses (i), i=1, . . . , M, the image sensor 490 may move a distance of less than the width 152 of a sensing element 150 of the image sensor 490 measured in a direction of said moving of the image sensor 490. In an embodiment, during each of the radiation pulses (i), i=1, . . . , M, the image sensor 490 may move a distance of less than one half of the width 152.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
shining a scene with radiation pulses (i), i=1, . . . , M, one pulse at a time, wherein M is an integer greater than 1;
for i=1, . . . , M, during the radiation pulse (i) and utilizing radiation of the radiation pulse (i), capturing, one by one, partial images (i,j), j=1, . . . , Ni of the scene with a same image sensor, wherein Ni, i=1, . . . , M are all integers greater than 1;
for i=1, . . . , M, generating an enhanced partial image (i) from the partial images (i,j), j=1, . . . , Ni by applying one or more super resolution algorithms to the partial images (i,j), j=1, . . . , Ni; and
stitching the enhanced partial images (i), i=1, . . . , M resulting in a stitched image of the scene.

2. The method of claim 1, wherein all Ni, i=1, . . . , M are the same.

3. The method of claim 1, wherein all Ni, i=1, . . . , M are greater than 100.

4. The method of claim 1, wherein for i=1, . . . , M, during the radiation pulse (i), the image sensor moves continuously with respect to the scene.

5. The method of claim 1, wherein the image sensor moves continuously with respect to the scene during a time period in which the image sensor captures all the partial images (i,j), i=1, . . . , M, and j=1, . . . , Ni.

6. The method of claim 5, wherein said moving of the image sensor with respect to the scene during the time period is at a constant speed.

7. The method of claim 1, further comprising arranging a mask such that for i=1, . . . , M, during the radiation pulse (i), (A) radiation of the radiation pulse (i) which is aimed at the scene but not aimed at active areas of the image sensor is prevented by the mask from reaching the scene, and (B) radiation of the radiation pulse (i) which is aimed at the scene and also aimed at the active areas of the image sensor is allowed by the mask to pass through the mask so as to reach the scene.

8. The method of claim 1, wherein during each of the radiation pulses (i), i=1, . . . , M, the image sensor moves a distance of less than a width of a sensing element of the image sensor measured in a direction of said moving of the image sensor.

9. The method of claim 1, wherein during each of the radiation pulses (i), i=1, . . . , M, the image sensor moves a distance of less than one half of said width.

10. The method of claim 1, wherein the image sensor comprises multiple radiation detectors.

11. An imaging system, comprising:
a radiation source configured to shine a scene with radiation pulses (i), i=1, . . . , M, one pulse at a time, wherein M is an integer greater than 1; and
an image sensor configured to, for i=1, . . . , M, during the radiation pulse (i) and utilizing radiation of the radiation pulse (i), capture one by one, partial images (i,j), j=1, . . . , Ni of the scene, wherein Ni, i=1, . . . , M are all integers greater than 1,
wherein the image sensor is configured to, for i=1, . . . , M, generate an enhanced partial image (i) from the partial images (i,j), j=1, . . . , Ni by applying one or more super resolution algorithms to the partial images (i,j), j=1, . . . , Ni, and
wherein the image sensor is configured to stitch the enhanced partial images (i), i=1, . . . , M resulting in a stitched image of the scene.

12. The imaging system of claim 11, wherein all Ni, i=1, . . . , M are the same.

13. The imaging system of claim 11, wherein all Ni, i=1, . . . , M are greater than 100.

14. The imaging system of claim 11, wherein for i=1, . . . , M, during the radiation pulse (i), the image sensor is configured to move continuously with respect to the scene.

15. The imaging system of claim 11, wherein the image sensor is configured to move continuously with respect to the scene during a time period in which the image sensor captures all the partial images (i,j), i=1, . . . , M, and j=1, . . . , Ni.

16. The imaging system of claim 15, wherein said moving of the image sensor with respect to the scene during the time period is at a constant speed.

17. The imaging system of claim 11, further comprising a mask arranged such that for i=1, . . . , M, during the radiation pulse (i), (A) radiation of the radiation pulse (i) which is aimed at the scene but not aimed at active areas of the image sensor is prevented by the mask from reaching the scene, and (B) radiation of the radiation pulse (i) which is aimed at the scene and also aimed at the active areas of the image sensor is allowed by the mask to pass through the mask so as to reach the scene.

18. The imaging system of claim 11, wherein during each of the radiation pulses (i), i=1, . . . , M, the image sensor is configured to move a distance of less than a width of a sensing element of the image sensor measured in a direction of said moving of the image sensor.

19. The imaging system of claim 11, wherein during each of the radiation pulses (i), i=1, . . . , M, the image sensor is configured to move a distance of less than one half of said width.

20. The imaging system of claim 11, wherein the image sensor comprises multiple radiation detectors.
